(19) United States
(12) Patent Application Publication
(10) Pub. No.: US 2026/0345866 A1
(43) Pub. Date: Nov. 5, 2026

(54) Orthopedic Implants with ROS-Responsive Antibiotic Coatings

(71) Applicant: University of Cincinnati, Cincinnati, OH (US)

(72) Inventors: John Robert Martin, Cincinnati, OH (US); Karina Ann Bruce, Findlay, OH (US); Dylan Widder Marques, Cincinnati, OH (US); Alan Joseph Fullenkamp, Beavercreek, OH (US)

(73) Assignee: University of Cincinnati, Cincinnati, OH (US)

(21) Appl. No.: 19/120,320

(22) PCT Filed: Oct. 11, 2023
(86) PCT No.: PCT/US2023/034931

§ 371 Date: Apr. 11, 2025

Related U.S. Application Data
(60) Provisional application No. 63/415,126, filed on Oct. 11, 2022.

(51) Int. Cl. A61L 27/54; A61L 27/34

(52) U.S. Cl. CPC A61L 27/54; A61L 27/34; A61L 2300/216; A61L 2300/252; A61L 2300/406; A61L 2420/02; A61L 2420/08

(57) ABSTRACT

An orthopedic implant is provided. The implant includes at least one coating on the surface of the implant. The coating has at least one drug and release of the drug is selectively triggered by inflammation. The coating may comprise one or more ROS-responsive polymers selected from the group consisting of poly(thioketal β-amino amide) (PTK-BAA), poly(thioacetal β-amino amide) (PTA-BAA), and poly(β-amino ester) (PBAE) chemistries.

### CROSS-REFERENCE TO RELATED APPLICATIONS

This application is a continuation of PCT Application No. PCT/US23/34931 filed Oct. 11, 2023, which claims the benefit of the filing date of U.S. Provisional Application No. 63/415,126 filed Oct. 11, 2022, the disclosures of which are incorporated by reference herein in their entirety.

### STATEMENT REGARDING FEDERALLY SPONSORED RESEARCH OR DEVELOPMENT

This invention was made with government support under grant W81XWH2210075 awarded by the Department of Defense-Peer Reviewed Medical Research Program (PRMRP). The government has certain rights in the invention.

### FIELD OF THE INVENTION

The present invention relates to orthopedic implants.

### BACKGROUND OF THE INVENTION

Bone injuries reconstructed from blast injuries or other trauma, especially when stabilized with orthopedic implants, are prone to serious bacterial infections. These pathologies are the most common complication behind orthopedic implant failure, costing the US healthcare system ~$2 billion annually, and often require numerous surgeries and months of patient morbidity for tissue remediation. Bacterial bone infections such as osteomyelitis are highly destructive pathologies that particularly affect service members and civilians following orthopedic trauma injuries. However, osteomyelitis poses a risk to all patients that receive orthopedic implants. It occurs in 28% of military personnel with combat-related open extremity wounds. It also occurs in 1-4% of individuals receiving a joint replacement. Further, bone injuries have a high risk for systemic spread of infection and a high risk for infection recurrence.

Current clinical strategies include a reactionary approach, where antibiotics are administered via IV infusion. If infection is unmanaged, it may require the administration of intravenous antibiotics over multiple weeks to months, surgical removal of the implant, extensive debridement of necrotic tissue and/or fixation of a new implant. Multi-stage revisions are associated with an over 500% increase in hospital expenditure.

Current technologies designed to locally deliver antibiotics to surgically-reconstructed bone injuries are limited by poorly controlled drug release kinetics and minimal responsiveness to the local healing environment. Therefore, a need still exists for an improved method to provided antibiotics to orthopedic implants.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention.

In an embodiment of the invention, an orthopedic implant is provided. The implant includes at least one coating on the surface of the implant. The coating has at least one drug and delivery of the drug is selectively triggered by inflammation of tissue located near the implant. In one embodiment, the drug comprises at least one antibiotic. In another embodiment, the antibiotic comprises p-anisaldehyde. In one embodiment, the coating comprises at least two antibiotic drugs. In another embodiment, the coating comprises p-anisaldehyde and a second antibiotic drug. In one embodiment, the second antibiotic drug is a cationic antibiotic. In another embodiment, the second antibiotic drug is selected from the group consisting of vancomycin, gentamicin and tetracycline. In one embodiment, the second antibiotic drug comprises vancomycin.

In one embodiment, the coating comprises one or more ROS-responsive polymers. In another embodiment, the one or more ROS-responsive polymers are selected from the group consisting of poly(thioketal β-amino amide) (PTK-BAA), poly(thioacetal β-amino amide) (PTA-BAA), and poly(β-amino ester) (PBAE) chemistries. In one embodiment, the one or more ROS-responsive polymers comprise one or more PTK-BAA chemistries. In another embodiment, the coating comprises multiple layers of film. In one embodiment, the multiple layers of film comprise at least four layers. In another embodiment, the multiple layers of film are formed using layer-by-layer (LbL) assembly. In one embodiment, the multiple layers of film alternate between polycation and polyanion layers.

In another embodiment of the invention, a method of preventing infection from an orthopedic implant is provided. The method involves implanting an orthopedic implant in a subject. The orthopedic implant includes at least one coating on the surface of the implant. The coating has at least one drug and release of the drug is selectively triggered by inflammation of the subject's tissue located near the implant. In one embodiment, the coating comprises one or more ROS-responsive polymers. In another embodiment, the one or more ROS-responsive polymers are selected from the group consisting of poly(thioketal β-amino amide) (PTK-BAA), poly(thioacetal β-amino amide) (PTA-BAA), and poly(β-amino ester) (PBAE) chemistries. In one embodiment, the coating comprises p-anisaldehyde and a second antibiotic drug. In another embodiment, the second antibiotic drug is a cationic antibiotic. In one embodiment, the second antibiotic drug is selected from the group consisting of vancomycin, gentamicin and tetracycline. In another embodiment, the second antibiotic drug comprises vancomycin. In one embodiment, the coating comprises multiple layers of film that are formed using layer-by-layer (LbL) assembly.

### BRIEF DESCRIPTION OF THE DRAWINGS

The objects and advantages of the disclosed invention will be further appreciated in light of the following detailed descriptions and drawings in which:

FIG. 1A is a schematic showing a standard orthopedic implant for fracture fixation with bacterial colonization and local bone tissue damage from infection.

FIG. 1B is a schematic showing an orthopedic implant with a responsive antibiotic coating according to the present invention. Further, the figure shows inflammation-triggered antibiotic delivery for improved bone healing.

FIG. 2A is a schematic of LbL nanolayered film fabrication using iterative adsorption of charged polyelectrolytes.

FIG. 2B is a graph showing that responsive LbL films form robust assemblies.

FIG. 2C is a graph showing that responsive LbL films have tunable drug loading.

FIG. 3 is a schematic showing that PTK-BAA and PTA-BAA polycations are amenable to LbL film formation and oxidative cleavage.

FIG. 4A is a graph showing that LbL films constructed with ROS-degradable PTK polymers/protein drugs selectively release encapsulated protein upon oxidation.

FIG. 4B is a graph showing that LbL films constructed with ROS-degradable PTK polymers/protein drugs strongly correlate with cellular bioactivity levels following film releaseate treatment.

FIG. 4C is a graph showing that LbL films constructed with ROS-degradable PTK polymers/protein drugs display “on-demand” protein release with pulsed ROS treatment.

FIG. 5A is a schematic showing an orthopedic implant and three potential coating conditions.

FIG. 5B is a graph showing the drug release kinetics of the second and third coating conditions of FIG. 5A.

FIG. 6 is a schematic of a synthesis pathway for a thioketal.

FIG. 7A is a graph showing an evaluation of bond persistence over time for various TK-pendant groups.

FIG. 7B is a graph showing bond persistence over time for DMPTK, LATK and PATK.

FIG. 7C is a graph showing bond persistence percentage for DMPTK, LATK and PATK.

FIG. 8 is a schematic showing a process for making LbL film constructed by alternating polycation and polyanion layers.

FIG. 9A is a schematic of the chemical structure of a PTK-PAA polycation.

FIG. 9B is a schematic of a synthesis pathway for a PTK-PAA polycation.

FIG. 10 is a schematic of the chemical structure of vancomycin.

FIG. 11 is a graph showing a vancomycin calibration curve.

FIG. 12A is an image of silicon wafers coated with a film according to the present invention.

FIG. 12B is an image of a custom stainless-steel bar coated with a film according to the present invention.

FIG. 12C is an image of a custom stainless-steel bar coated with a film according to the present invention.

FIG. 13 is a schematic showing a femoral defect model.

FIG. 14 is an image of stainless-steel plates according to the present invention attached to a mouse femur.

### DEFINITIONS

As used herein, the term “about,” when referring to a value or to an amount of mass, weight, time, volume, pH, size, concentration, or percentage, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

As used herein, “ROS-responsive” means the system responds to an increase in naturally occurring biological stimuli (known as reactive oxygen species (ROS)) that is a hallmark of inflammation, disease states, and infection.

### DETAILED DESCRIPTION OF THE INVENTION

One or more specific embodiments of the present invention are described below. In an effort to provide a concise description of these embodiments, all features of an actual implementation may not necessarily be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

An ideal treatment for fracture-associated osteomyelitis is prophylactic in nature, preventing or eliminating local bacterial infections before they compromise regenerating bone tissue and necessitate extensive surgical interventions. While local antibiotic delivery from implants has been demonstrated, previous technologies predominantly feature “top-down” engineering strategies that rely on pre-determined drug release rates that often do not match bacterial infection progression or recurrence events.

Instead, the delivery platform of the present invention directly links localized, prophylactic antibiotic release to an infection-specific stimulus to prolong therapeutic delivery profiles. This represents a significant shift from conventional antibiotic delivery schemes and has the potential to truly personalize therapies for injured service members and patients with highly variable disease progression profiles. To directly tie local antibiotic release to the recognition of bacterial infection, the present invention uses nanoscale drug coatings, fabricated on the surface of orthopedic implants, that are selectively triggered by inflammation. This responsive system discharges antibacterial therapies only when needed, thereby significantly extending the therapeutic delivery window of local antibiotic treatments by creating “on-demand” drug release for combating pathogen recurrences. This responsive technology offers an innovative strategy to better treat intermittent and recurrent bone infections without the limitations of passive, short-lived drug release technologies.

In one embodiment, the present invention involves an orthopedic implant comprising at least one drug coating on the surface of the implant, where the drug is selectively triggered by inflammation of tissue located near the implant. In one embodiment, the drug coating comprises at least one antibiotic. In another embodiment, the drug coating comprises p-anisaldehyde. In one embodiment, comprises p-anisaldehyde and vancomycin.

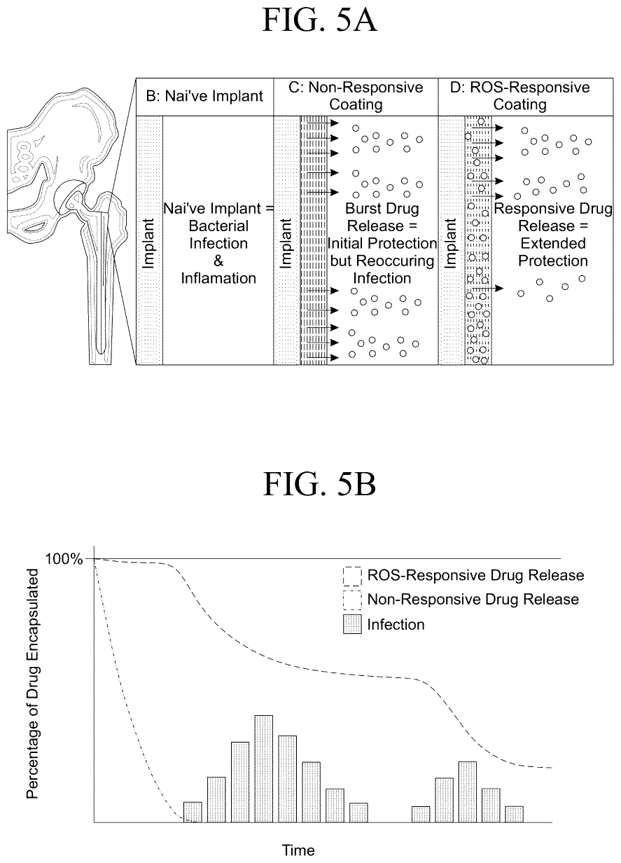

The present invention involves the development of drug loaded antibacterial coatings for orthopedic implants that are ROS responsive. The system of the present invention provides an “on-demand” drug delivery with extended-release kinetics. The system mitigates bacterial infection recurrence. The ROS (reactive oxygen species) responsive materials are naturally occurring biological mediators. Elevated levels are seen in infection, disease states and inflammation. Referring to FIG. 5A, a schematic shows an orthopedic implant and three potential coating conditions. First, the implant may be a naïve implant with no coating. This implant has a greater chance of bacterial infection and inflammation. Second, the implant may have a non-responsive coating. Such a coating will have a burst drug release. This type of release will provide initial protection, but still presents a chance of reoccurring infection. Third, the implant may have a coating according to the present invention. This coating is ROS-responsive. Such a coating provides a responsive drug release, allowing extended protection from infection. Referring to FIG. 5B, a graph shows the drug release kinetics of the second and third coating conditions. The ROS-responsive coating maintains a higher level of encapsulated drug over time.

Besides creating vancomycin-releasing films with ROS-responsive polymers, the specific strategy of directly incorporating aldehyde-containing antimicrobial compounds into the polymer's degradable linker is particularly innovative as it significantly increases drug payload incorporation while precisely linking antibiotic discharge to polymer chain scission. Moreover, the described LbL thin film assembly techniques can create robust coatings using a variety of drug compounds, leaving this technology platform ideally situated for utilization in other medical pathologies that require localized pharmacological interventions.

### Osteomyelitis Pathogenesis and Recurrence Events

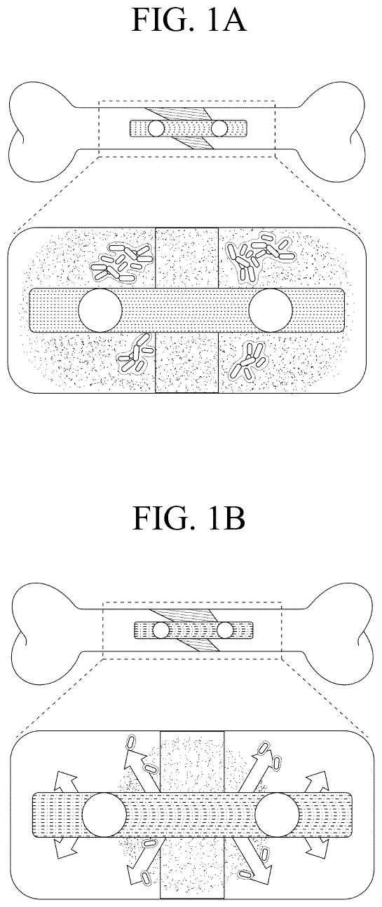

Osteomyelitis, or bacterial infection of bone, marrow, or surrounding soft tissues, remains a destructive pathology for patients with surgically reconstructed bone. In particular, blast injuries suffered by armed forces members during combat tours often result in open, complex fractures that are susceptible to bacterial colonization and infection. Fractures requiring internal fixation are also highly prone to developing osteomyelitis either from initial bacterial seeding of the implant or transmission during hospital care as denoted in FIG. 1A. The figure shows recurrent bacterial infection of implant-stabilized bone injuries. These infections can lead to bone tissue necrosis and often require multi-stage revision, first removing the initial implant and compromised bone, administering weeks of systemic antibiotics to eliminate infection, and then performing a final reconstructive procedure. Unfortunately, these efforts are often insufficient for curative treatment as a recent study of combated-wounded US military personnel found over 30% of osteomyelitis patients suffered infection recurrence after initial therapies. A number of bacteria are thought to trigger osteomyelitis, though opportunistic gram-positive Staphylococcus aureus bacteria are found in ~75% of documented infections. S. aureus are sensitive to a number of antibiotics, including vancomycin, gentamicin, and other cationic antibiotics, though systemic administration of antimicrobial drugs has also helped spur the rise of drug-resistant strains such as methicillin-resistant S. aureus (MRSA).

### Local Antibiotic Delivery From Orthopedic Implants

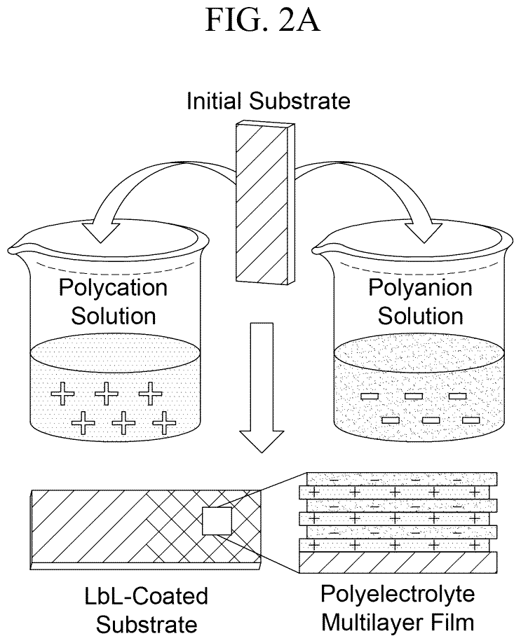

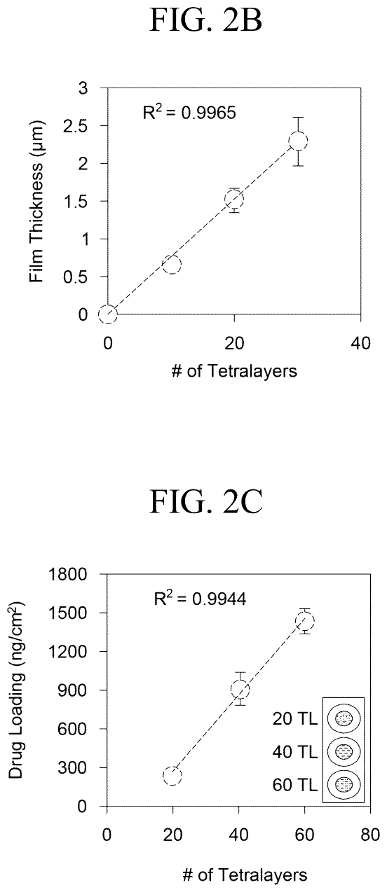

Due to osteomyelitis's highly damaging impact and the desire to maximize therapeutic localization for preventing antimicrobial resistance development, a number of strategies have pursued local delivery of antibiotics from orthopedic materials to combat these bone infections. Most notable are antibiotic-loaded polymeric bone cements, which have been used clinically to deliver tobramycin, gentamicin, and vancomycin for infection prevention in orthopedic reconstructions. However, these cements offer poor control over antibiotic release kinetics and often discharge the bulk of their drug payload within one week of implantation. To gain more control over antimicrobial delivery, the present invention directly coats orthopedic hardware with tunable, degradable antibiotic films using layer-by-layer (LbL) deposition techniques as outlined in FIG. 2A. These robust electrostatic surface coatings effectively encapsulate therapeutic drug compounds under mild aqueous fabrication conditions and do not alter or degrade the underlying material substrate. Importantly, as demonstrated in tests with protein drugs, LbL assemblies also allow for highly tunable film growth (FIG. 2B) and drug loading (FIG. 2C) with increasing layer depositions. To foster release of film-encapsulated therapeutics, biodegradable polymers with tunable erosion profiles are often incorporated into the assemblies. Hydrolytically degradable poly(β-amino ester) (PBAE) polymers have previously been employed to control antibiotic release from LbL coatings, though crucially these formulations release over 75% of their drug payload within 48 h. These abbreviated drug release kinetics highlight the potentially limited effectiveness of hydrolysis-driven drug delivery for combatting advanced infections.

To address these concerns, recent efforts have pursued LbL coatings with “smart” therapeutic delivery that enables selective drug release in response to tissue-produced signals such as reactive oxygen species (ROS). Specific ROS-triggered release of therapeutic proteins from polymeric LbL coatings, both in vitro and in vivo, has been demonstrated, validating the feasibility of tissue-responsive drug delivery from LbL films. However, oxidation-triggered antimicrobial delivery from LbL films has not been previously reported. The present invention creates a sustained, “on-demand” antibiotic delivery system that selectively releases antimicrobial compounds from surface-coated orthopedic implants in response to elevated ROS levels as illustrated in FIG. 1B. The figure shows an inflammation-responsive implant coating according to the present invention for on-demand, prophylactic antibiotic delivery to improve bone healing. Crucially for this antimicrobial application, local tissue concentrations of ROS spike during bacterial infection as inflammatory ROS-producing immune cells attempt to quell bacterial proliferation, thus making local ROS concentration a precise signal for initiating local drug delivery. By releasing the therapeutic payload only when needed, the drug delivery window can be drastically extended while also limiting any negative effects on bone growth or osteogenesis associated with excess concentrations of antibiotics.

### Antibiotics

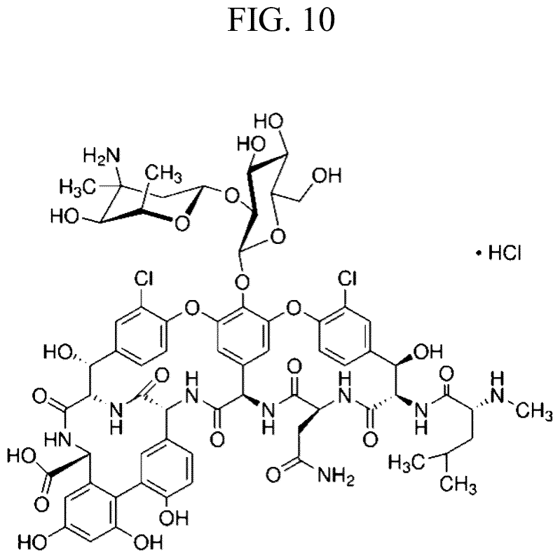

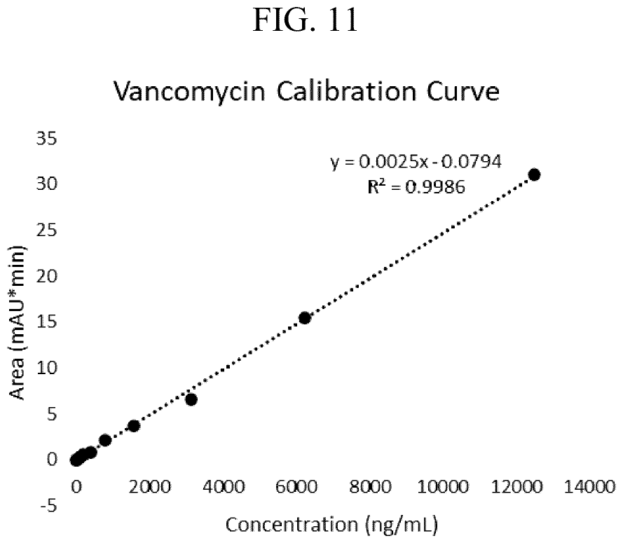

Antibiotics that are useful in the present invention include vancomycin (FIG. 10), gentamicin and tetracycline. Other broad-spectrum antibiotics may also be useful. In one embodiment, the antibiotics of the present invention have cationic properties. They are typically used clinically. In addition, HPLC can be used to quantify drug loading and release kinetics. For example, vancomycin can be quantified using HPLC. Films according to the present invention are incubated in in various doses of ROS. The film is then removed from solution and the releasate sample is run on HPLC. FIG. 11 shows a current calibration curve for vancomycin from 16 ng/mL-12,500 ng/mL. The curve can be extended to lower concentrations.

### Antibiotic Delivery

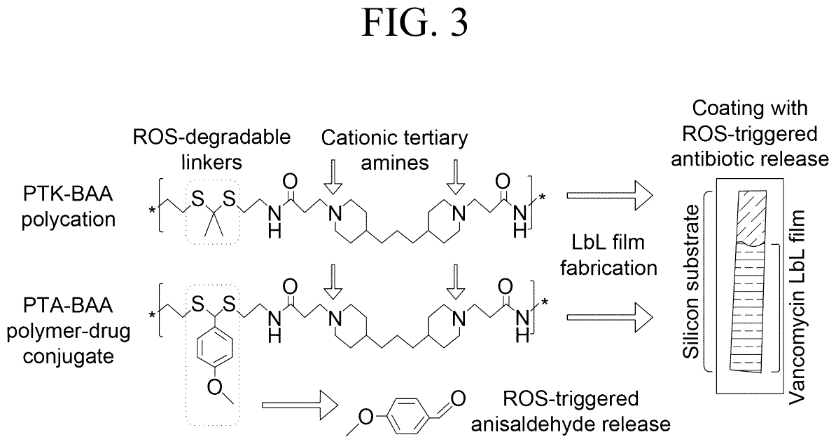

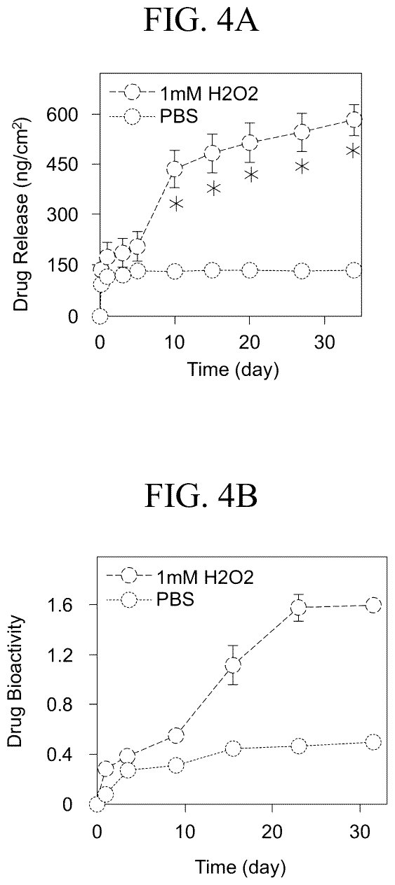

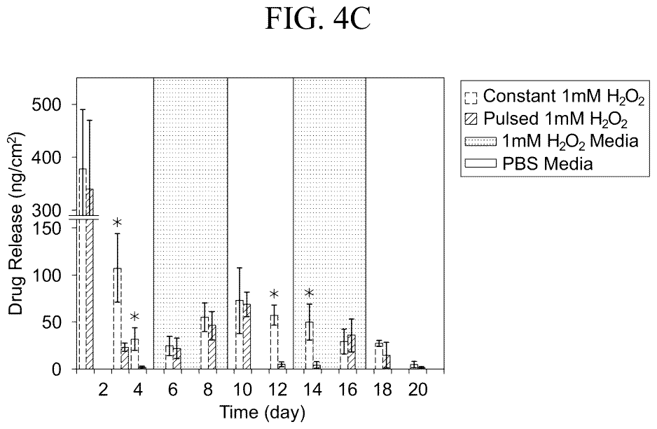

The on-demand antibiotic delivery provided by the inflammation-responsive coatings on the surface of orthopedic implants of the present invention will prolong local drug delivery and mitigate bacterial infection recurrence in bone injuries. Local antibiotic delivery from bone implants has shown promise in mitigating bacterial colonization and infection in orthopedic procedures, though current iterations are limited in their duration of effectiveness due to rapid release of drug payloads. To that end, the present invention creates a sustained, “on-demand” antibiotic delivery system that selectively releases antimicrobial compounds in response to the elevated levels of ROS produced by bacteria-inflamed tissues (FIG. 1B). Formed on the surface of clinically-applicable fracture fixation plates, one embodiment of the present system employs conformal LbL coatings (FIG. 2A) containing S. aureus killing vancomycin antibiotics and ROS-sensitive polymers (FIG. 3) that mediate drug discharge upon inflammation-mediated oxidation. In experiments with model protein drugs, these ROS-sensitive LbL films selectively discharged therapeutics under oxidative conditions (FIG. 4A), facilitated cellular osteogenic bioactivity corresponding with drug release kinetics from films (FIG. 4B), and demonstrated “on-demand” protein release with pulsatile ROS treatment (FIG. 4C). The present invention leverages these responsive drug coatings for inflammation-triggered antibiotic delivery, evaluating drug release and efficacy in vitro. These systems have also been used to combat recurrent S. aureus infections in a rodent bone defect model. These tests establish the feasibility of responsive, preventative antibiotic delivery in orthopedic repair and introduce a robust strategy for achieving sustained antimicrobial delivery in regenerative applications.

### Thioketals

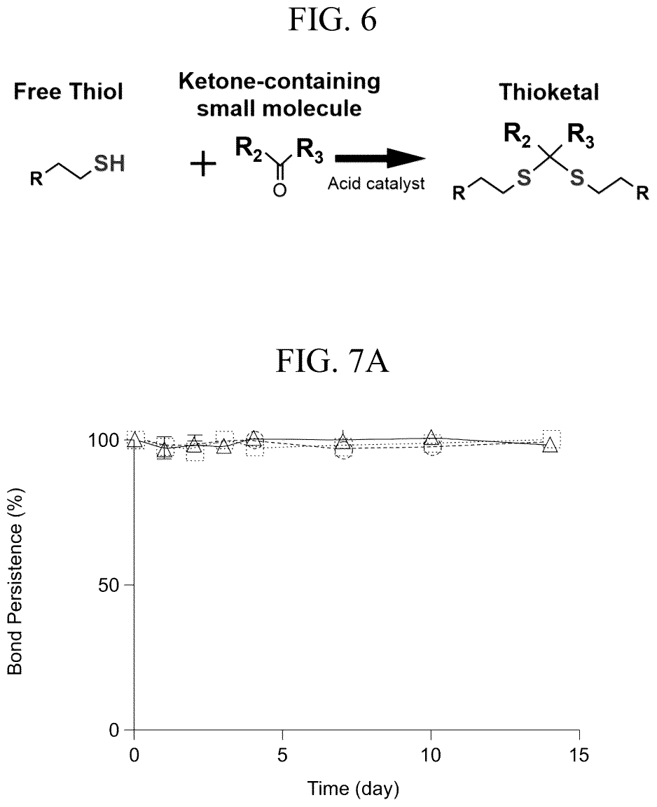

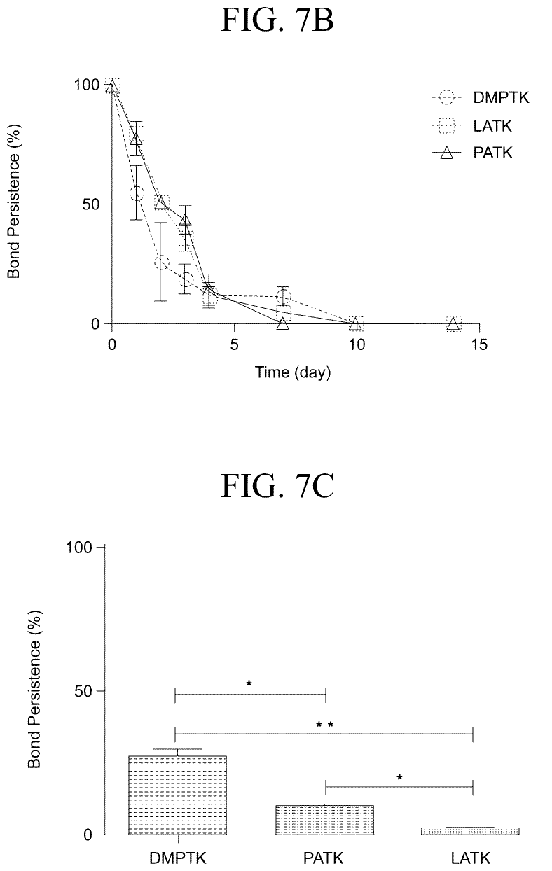

In one embodiment, the ROS-responsive polymers of the present invention are thioketals (TK) that are selectively degradable via ROS. A synthesis pathway is shown in FIG. 6. These polymers are stable in aqueous environments. Evaluation of various TK-pendant groups are shown in FIG. 7A (bond persistence over time), FIG. 7B (bond persistence over time for DMPTK, LATK and PATK), and FIG. 7C (bond persistence percentage for DMPTK, LATK and PATK).

### Antibiotic Loading in ROS-Responsive LbL Coatings

Charged poly(thioketal β-amino amide) (PTK-BAA) polymers are first synthesized using established methods and characterized 1H nuclear magnetic resonance (NMR) to confirm successful polymerization. These polymers feature ionizable tertiary amines alongside ROS-degradable thioketal groups (FIG. 3), respectively making these materials amenable to electrostatic LbL assembly and selective oxidation-mediated film disassociation.

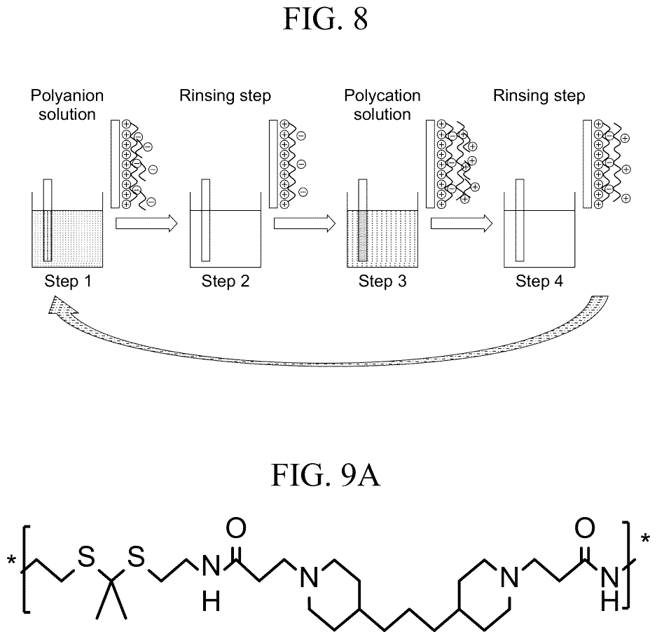

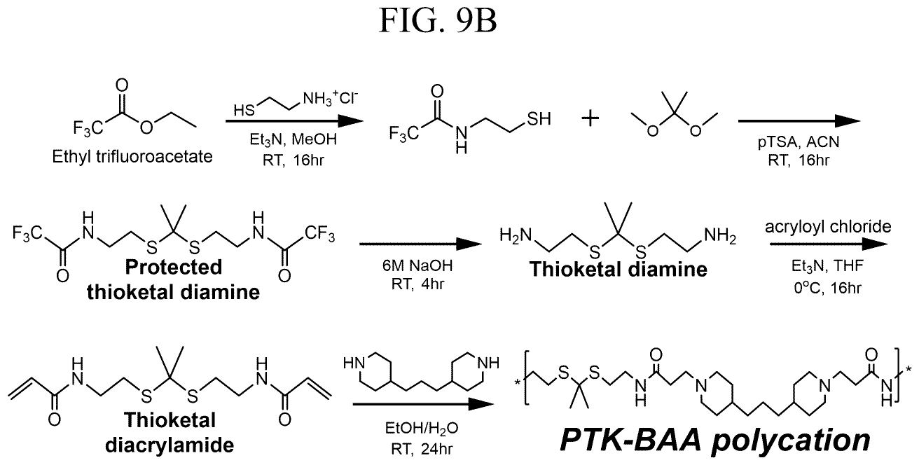

In one embodiment, the LbL film coatings of the present invention are constructed by alternating polycation and polyanion layers (see FIG. 8). For example, the layers may be arranged as follows: polycation layer (ROS-responsive polymer), polyanion layer (Poly(acrylic acid)), cation layer (Antibiotic drug) and polyanion layer (Poly(acrylic acid)). In one embodiment, the ROS-responsive polymer used as the polycation layer is PTK-BAA polycation (FIG. 9). FIG. 9B shows a synthesis pathway for the PTK-BAA polycation.

Vancomycin-loaded LbL films are constructed on model stainless steel plates using alternating adsorptions of cationic PTK-BAA, anionic poly(acrylic acid) (PAA), cationic vancomycin, and then PAA again in a repeating tetralayer architecture. Vancomycin has previously been successfully incorporated into hydrolysis-sensitive LbL assemblies, and was successfully complexed with the PTK-BAA polycation to generate robust LbL films (40 tetralayers) as pictured in FIG. 3. LbL assembly conditions (solution pH, polyelectrolyte concentration, dip durations) can be adjusted to maximize per-cycle vancomycin loading.

As demonstrated in LbL films constructed with PTK-BAA polymers and protein therapeutics (FIG. 2C), vancomycin loading vs. tetralayer depositions can be quantified to determine tunable antibiotic dosing in the coatings. Total vancomycin loading can be quantified using high-performance liquid chromatography (HPLC) following whole-film disassociation in high salt conditions. Vancomycin loading of 10 μg per cm² of film surface area is achievable with these LbL systems and provides potent antimicrobial activity. Additionally, a hydrolytically degradable poly(β-amino ester) (PBAE) polycation can be similarly polymerized and used to generate control vancomycin LbL coatings with non-responsive drug release.

### Antibiotic Loading Via PTA Polymer-Drug Conjugates

Concurrent with the work described above, a poly(thioacetal β-amino amide) (PTA-BAA) polymer-drug conjugate was synthesized and evaluated as an LbL film constituent (FIG. 3). Compared to the PTK-BAA coatings which solely release vancomycin, PTA-BAA films more strongly inhibit bacterial growth since they deliver both film-loaded vancomycin alongside a polymer-conjugated antibiotic upon oxidative triggering. Thioketal and thioacetal bonds are both sensitive to oxidation, though polymeric drug delivery systems featuring either of these ROS cleavable linkers have primarily relied on simple polymer chain degradation and loss of electrostatic or hydrophobic interactions with drug compounds to facilitate release. Although historically formed with simple precursors such as acetone or formaldehyde, thioketals and thioacetals can also be respectively synthesized from more complex compounds featuring unprotected ketone or aldehyde groups. The strategy disclosed herein covalently incorporates a drug molecule directly into the degradable linker, so oxidation of the thioketal or thioacetal group not only cleaves the polymer chain but liberates the intact bioactive drug compound. A comparable strategy has been recently pursued using aldehyde-based antimicrobials though by employing similarly synthesized polymers with pH-sensitive acetal linkers instead of thioacetals.

The present invention modifies this approach by delivering the antimicrobial compound p-anisaldehyde from thioacetal polymer-drug conjugates via ROS-mediated drug liberation (FIG. 3). First, p-anisaldehyde is used to generate PTA-BAA polymers using previously described protocols before confirming polymerization by GPC and NMR. Next, PTA-BAA degradation and p-anisaldehyde release kinetics following treatment with escalating doses of the model ROS model hydrogen peroxide (0, 0.1, 1, 10, 100 mM H₂O₂) is determined by NMR. Additionally, following optimization strategies established with the PTK-BAA/vancomycin films, the PTA-BAA polycations is similarly complexed into LbL assemblies with vancomycin to create dually-loaded coatings. Per-layer drug encapsulation, along with drug loading vs. layer depositions, can be optimized and quantified as described above.

### Outcomes

Based on past reports with similar systems and the constructed PTK/vancomycin films shown in FIG. 3, both PTK-BAA and PTA-BAA polymers will successfully form stable vancomycin LbL films, feature selective and dose-dependent ROS-triggered drug release, and to cause minimal toxicity to mammalian cells. Also, PTA-BAA films elicit greater antimicrobial effect due to their co-delivery of p-anisaldehyde with vancomycin upon oxidative polymer degradation, and can be carried forward for in vivo testing. In addition, as demonstrated previously, ROS-responsive coatings will significantly prolong drug delivery in vivo compared against the conventional non-responsive formulations. Moreover, drug release from the oxidation-sensitive films rapidly increase upon bacterial re-infection while the non-responsive coatings have exhausted their antibiotic supply by the 14 day point. Finally, the ROS-responsive drug coatings will prevent prolonged bacterial persistence in the bone defects while uncoated and non-responsive films will retain S. aureus infection over 8 weeks. Also, that bacterial elimination mediated by the triggerable drug films will consequently lead to increased bone volumes via μCT and improved bone tissue morphology by histology.

### Alternative Strategies

Alternate methods for assembling the PTK-BAA/vancomycin LbL films of the present invention can be used. Additionally, the molecular weight of the polyanion used in LbL film construction can be modulated to adjust antibiotic release kinetics upon ROS triggering, presenting a simple strategy for tuning oxidative sensitivity in these systems. Regarding testing, the Xen29 S. aureus strain has been successfully used to elicit osteomyelitis in rat long bone defects. Employing an antibiotic-coated bone plate will effectively recapitulate a relevant clinical scenario for stabilizing infection-prone open fractures, and bone plates are commonly used for fracture fixation in rat models. Alternatively, a press-fit implant with the respective antibiotic coating can be directly inserted into the surgical defect for fixation-free administration. Dosing for the antibiotic payload can be modulated by increasing vancomycin layers in the LbL assembly (FIG. 2C) or by changing the LbL's polyanion.

### EXAMPLES

### Example 1—ROS-Responsive Release and Antimicrobial Activity of Film-Discharged Therapeutics

Vancomycin LbL films constructed with three polycations (ROS-degradable PTK-BAA, ROS-degradable and anisaldehyde-releasing PTA-BAA, and hydrolytically-degradable PBAE) are formed on the surface of stainless steel plates at matching vancomycin doses. As guided by methodologies from similar experiments with PTK-based LbL films (FIG. 4A), coated samples are incubated at 37 °C in escalating doses of H₂O₂ (0, 0.1, 1, 10, 100 mM) dissolved in PBS at pH 7.4. To demonstrate “on demand” therapeutic delivery as previously demonstrated (FIG. 4C), drug-loaded films are also incubated in pulsed doses of 1 mM H₂O₂ to mimic infection recurrence events that ramp local tissue inflammation up or down. For all antibiotic release studies, releaseate are collected every 2 days to measure vancomycin and panisaldehyde concentrations with HPLC. Next, a microdilution procedure to assess antibiotic bioactivity with Staphylycoccus aureus bacteria (ATCC) is performed. Drug releaseate samples are first lyophilized to remove residual cytotoxic H₂O₂, then resuspended in PBS at 1, 2, 3, and 4-fold dilutions, and administered with exponentially growing S. aureus bacteria (105 CFU/mL in cation-adjusted Mueller Hinton broth) in a 96-well plate for 16 h at 37 °C. Comparing against both negative (no bacteria) and positive (PBS treatment) controls, S. aureus inhibition by film-released antibiotics is assessed using a LIVE/DEAD BacLight Bacterial Viability Assay (Integra Biosciences). To ensure all materials and formulations are minimally cytotoxic to mammalian cells, naïve film constituents and drug-loaded films are incubated with murine MC3T3-E1 osteoblasts (ATCC) in standard culture media at 37 °C for 24 h and quantified for number of viable cells using a Cell TiterGLO assay (Promega).

### Example 2—In Vivo Antibiotic Release and ROS Production

To assess infection-triggered release of vancomycin from coated orthopedic hardware in vivo, drug-loaded bone plates are surgically fixed on Staphylococcus aureus infected bone injuries created in Sprague-Dawley rat femurs. A formulation with fluorophore-functionalized vancomycin is coated into LbL films deposited on the surface of 316L stainless steel bone plates (3-hole plate, 15 mm length, Orthomed). Films feature either ROS-responsive lead-candidate PTK-BAA/PTA-BAA polymers or hydrolysis-sensitive (non-ROS responsive) PBAE polycations. 1.5 mm diameter bone defects are created in each animal's femur with a saline-irrigated dental drill before inoculating with S. aureus (5×10⁵ CFU) and screwing in the coated plate to cover the injury site (n=6 animals per treatment group, half male half female cohort). Release of fluorescently-tagged vancomycin from the ROS responsive and non-responsive control coatings are non-invasively monitored by fluorescent IVIS imaging (Perkin Elmer). Additionally, the luminescent ROS-reporter molecule luminol are administered to the animals to measure local ROS production kinetics at the infected bone injury sites via luminescent IVIS imaging. To mimic a bacterial recurrence event, after 14 days the bone defects are re-inoculated with bacteria and monitoring is continued for vancomycin release and ROS production by IVIS until day 35.

### Example 3—Bacterial Recurrence Protection and Bone Healing in Rat Femoral Defects

Employing the same infected femoral defect model described above but instead using a luminescent-reporter line of S. aureus (Xen29, Perkin Elmer) for monitoring in vivo bacterial persistence, the three bone plate formulations (uncoated, PBAE/vancomycin coated, and PTK or PTA/vancomycin coated) are implanted in infected rat femurs for 8 weeks (n=6 animals per group). Non-invasive measurements of bacterial survival are quantified by IVIS every 3 days. At day 14 post-surgery, injury sites are re-opened, swabbed, and cultured ex vivo to assess S. aureus persistence at the wound site. The bone defects are also re-inoculated with bacteria at day 14 to mimic an infection recurrence event. To monitor temporal bone regeneration, non-invasive microcomputed tomography (μCT) is also used at weeks 3, 5, and 8. Animals are humanely euthanized at week 8 and analyzed via swabbing and bacterial plating to assess S. aureus persistence, scanning electron microscopy of bone explants to gauge biofilm formation, and histology to assess bone tissue morphology via trichrome staining.

### Example 4—Film Application

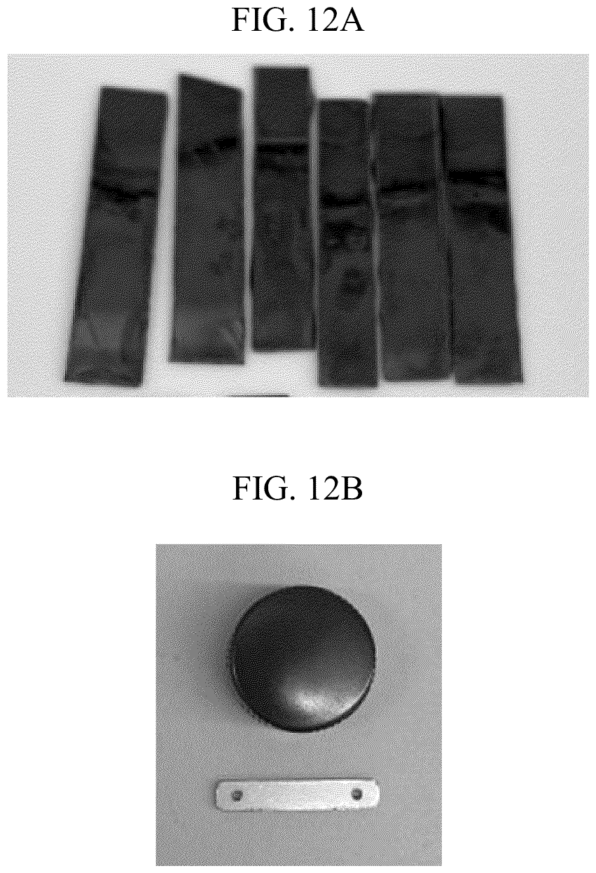

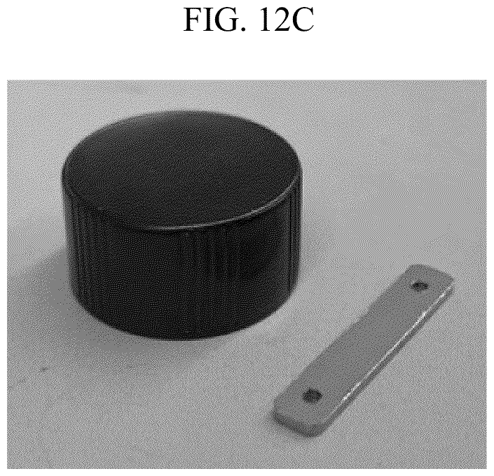

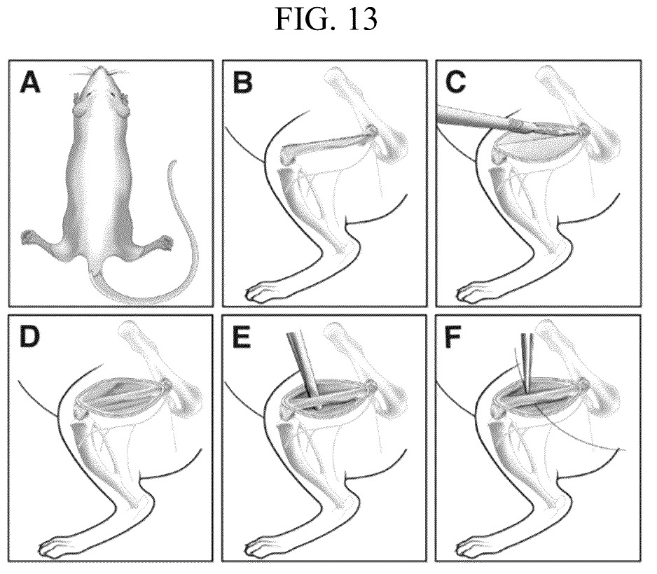

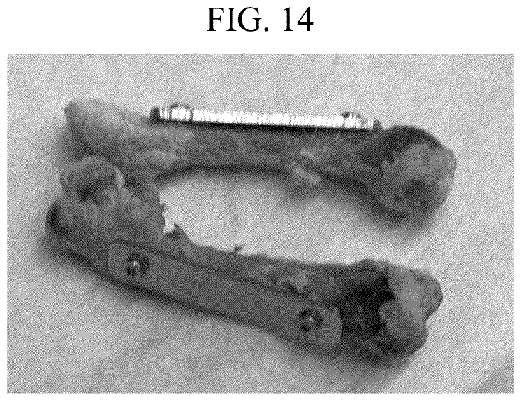

An embodiment of the film of the present invention was constructed. First, a model of the film was formed on silicon wafers (see FIG. 12A). The silicon wafers were plasma treated to provide initial surface charge. In vitro evaluation of drug loading, release, and cytotoxicity was conducted. Next, a custom stainless-steel plate was coated with the film (see FIGS. 12B and 12C). In vivo femoral reconstruction modeling was conducted using the stainless-steel plate. FIG. 13 shows the femoral defect model and FIG. 14 is an image of the attached stainless-steel plates.

While all the invention has been illustrated by a description of various embodiments, and while these embodiments have been described in considerable detail, it is not the intention of the Applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the Applicant's general inventive concept.

What is claimed is:

1. An orthopedic implant comprising at least one coating on the surface of the implant; wherein the coating comprises at least one drug and the drug is selectively triggered by inflammation.

2. The implant of claim 1 wherein the drug comprises at least one antibiotic.

3. The implant of claim 2 wherein the antibiotic comprises p-anisaldehyde.

4. The implant of claim 1 wherein the coating comprises at least two antibiotic drugs.

5. The implant of claim 4 wherein the coating comprises p-anisaldehyde and a second antibiotic drug.

6. The implant of claim 5 wherein the second antibiotic drug is selected from the group consisting of vancomycin, gentamicin and tetracycline.

7. The implant of claim 5 wherein the second antibiotic drug comprises vancomycin.

8. The implant of claim 1 wherein the coating comprises one or more ROS-responsive polymers.

9. The implant of claim 8 wherein the one or more ROS-responsive polymers are selected from the group consisting of poly(thioketal β-amino amide) (PTK-BAA), poly(thioacetal β-amino amide) (PTA-BAA), and poly(β-amino ester) (PBAE) chemistries.

10. The implant of claim 8 wherein the one or more ROS-responsive polymers comprise one or more PTK-BAA chemistries.

11. The implant of claim 1 wherein the coating comprises multiple layers of film.

12. The implant of claim 11 wherein the multiple layers of film comprise at least four layers.

13. The implant of claim 11 wherein the multiple layers of film are formed using layer-by-layer (LbL) assembly.

14. The implant of claim 11 wherein the multiple layers of film alternate between polycation and polyanion layers.

15. A method of preventing infection from an orthopedic implant comprising implanting an orthopedic implant in a subject, the orthopedic implant comprising at least one coating on the surface of the implant; wherein the coating comprises at least one drug and the drug is selectively triggered by inflammation.

16. The method of claim 15 wherein the coating comprises one or more ROS-responsive polymers.

17. The method of claim 16 wherein the one or more ROS-responsive polymers are selected from the group consisting of poly(thioketal β-amino amide) (PTK-BAA), poly(thioacetal β-amino amide) (PTA-BAA), and poly(β-amino ester) (PBAE) chemistries.

18. The method of claim 16 wherein the coating comprises p-anisaldehyde and a second antibiotic drug.

19. The method of claim 17 wherein the wherein the second antibiotic drug is selected from the group consisting of vancomycin, gentamicin and tetracycline.

20. The method of claim 15 wherein the coating comprises multiple layers of film that are formed using layer-by-layer (LbL) assembly.
